(19) United States
(12) Patent Application Publication
(10) Pub. No.: US 2006/0263423 A1
(43) Pub. Date: Nov. 23, 2006

(54) Product and process for increasing compactibility of carbohydrates

(75) Inventors: Gary T. Norman, Middletown, DE (US); Cecil W. Propst, Norton Shores, MI (US)

Correspondence Address: MORGAN, LEWIS & BOCKIUS LLP, 1701 MARKET STREET, PHILADELPHIA, PA, 19103-2921, US

(73) Assignee: SPI Pharma, Inc.

(21) Appl. No.: 11/413,674

(22) Filed: Apr. 27, 2006

Related U.S. Application Data
(63) Continuation of application No. PCT/US04/35982, filed on Oct. 28, 2004.
(60) Provisional application No. 60/515,330, filed on Oct. 28, 2003.

(51) Int. Cl. A61K 9/20; A23G 3/00

(52) U.S. Cl. 424/464; 426/660; 264/109

(57) ABSTRACT

The present invention includes a method for preparing a highly compactible carbohydrate product, and the product itself. In one embodiment, a composition according to the present invention includes polyols.

### FIELD OF THE INVENTION

The present invention relates to a process for producing a highly compactible composition including at least two carbohydrates. The present invention also relates to the highly compactible carbohydrate product, and a pharmaceutical composition comprising the product.

### BACKGROUND

Carbohydrates are a common ingredient in pharmaceutical compositions as fillers for preparing solid dosage forms, such as tablets. These carbohydrate solid dosage forms are generally prepared using various processes, such as spray-drying, fluid bed granulation, and conventional wet granulation.

### BRIEF SUMMARY OF THE INVENTION

The present invention includes methods for preparing a highly compactible carbohydrate product. The method includes the steps of blending at least a first carbohydrate and a second carbohydrate, wherein the first carbohydrate has a melting point that is higher than the second carbohydrate; melting the second carbohydrate over the first carbohydrate to obtain a highly compacted product; drying the product; and screening the dried product to a desired particle size.

The present invention also includes carbohydrate compositions that include at least a first carbohydrate and a second carbohydrate, wherein the first carbohydrate has a melting point which is greater than said second carbohydrate, the second carbohydrate is uniformly melted over the first carbohydrate.

Finally, the present invention includes pharmaceutical compositions that include a carbohydrate composition of the present invention, and at least one of an active ingredient, a lubricant, a flavor, or a color.

The present invention provides a method for producing a carbohydrate composition that is highly compactible and has decreased ejection forces, thereby decreasing the tendency to laminate during tableting.

### BRIEF DESCRIPTION OF THE DRAWINGS

FIG. 1 is a graph presenting compactibility of various compositions containing different grades and forms of mannitol, sorbitol, mannitol and sorbitol, and calcium carbonate, all with magnesium stearate as a lubricant, where the black circle indicates one embodiment of a composition of the present invention.

FIG. 2 is a graph representing ejection forces for various grades and forms of mannitol, sorbitol, mannitol and sorbitol, and calcium carbonate, all with magnesium stearate as a lubricant, where the black circle indicates one embodiment of a composition of the present invention.

FIG. 3 is a graph representing compactibility of mannitol (asterisk), mannitol with 3.4% sorbitol (black diamond), and mannitol with 5% sorbitol (open square), according to the present invention.

FIG. 4 is a schematic representation of an extruder and an extrusion process for use in the present invention.

FIGS. 5A and 5B are schematic representations of a cross section of tablet. A cross-section of the particle is also depicted (FIG. 5A), including the individual fibers of sorbitol-coated mannitol (FIG. 5B).

### DETAILED DESCRIPTION

The present invention will be better understood with reference to certain definitions, provided below.

### DEFINITIONS

As used herein, the term “about” will be understood by persons of ordinary skill in the art and will vary to some extent on the context in which it is used. If there are uses of the term which are not clear to persons of ordinary skill in the art given the context in which it is used, “about” shall mean up to plus or minus 10% of the particular value.

As used herein, the term “compactibility” means the loss in volume of a powder being compacted with subsequent gain in mechanical strength of the solid dosage form. As used herein, the terms “solid dosage form,” “tablet,” and “solid preparation” are used synonymously within the context of the present invention. These terms should be construed to include a compacted or compressed composition obtained by compressing or otherwise forming the composition to form a solid having a defined shape.

### DETAILED DESCRIPTION

The present invention includes a method for preparing a highly compactible carbohydrate product. In one embodiment, the method includes blending at least a first carbohydrate (i.e., a higher melting point carbohydrate) and a second carbohydrate (i.e., a lower melting point carbohydrate), wherein the first carbohydrate has a melting point that is higher than the second carbohydrate, melting the second carbohydrate over the first carbohydrate to create a highly compactible product, drying the product, and screening the product for a desired particle size. Desirable particle sizes range from about 50 microns to about 800 microns, more preferably about 75 microns to about 590 microns, and most preferably, about 100 microns to about 420 microns. In one embodiment of the present invention, the mean particle size is in the range of about 250 microns to about 500 microns.

In one embodiment, the method includes blending at least a first carbohydrate and a second carbohydrate, wherein the first carbohydrate has a melting point that is higher than the second carbohydrate; extruding the carbohydrate blend through an extruder, creating an extrudate; drying the extrudate; and screening the dried extrudate to a desired particle size, wherein the temperature inside said extruder reaches the melting point of the second carbohydrate but not the first carbohydrate.

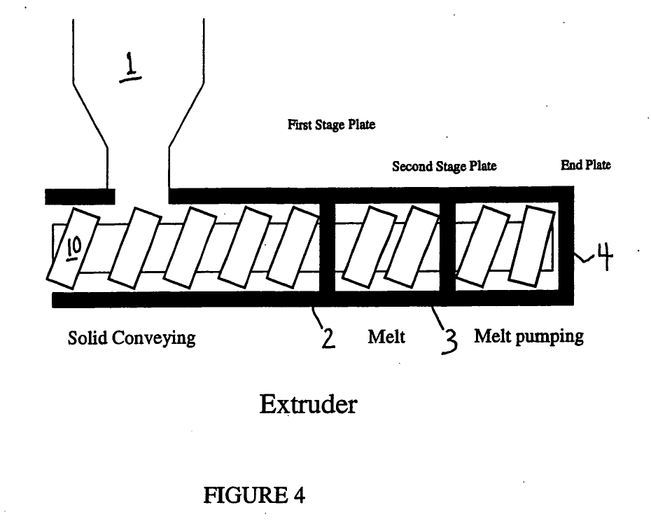

FIG. 4 is a schematic representation of an embodiment of a method of the present invention. The angled rectangles represent screw threads 10. In one embodiment, screw threads 10 have a pitch angle that increases from right to left in FIG. 4. The solid vertical rectangles indicate a first die plate 2, a second die plate 3, and an end die plate 4. In one embodiment, the first and second carbohydrates are fed by one or more feeder lines into a hopper 1, preferably in a rated manner (i.e., the first and second carbohydrates are fed into the hopper 1 in a manner that maintains a specific ratio between the first and second carbohydrates), along with a small amount of water. In one embodiment, blending occurs as the carbohydrates move along the screw path (prior to extrusion through the first die plate 2) as the screw thread pitch of the extruder changes, and/or by pressure mixing caused by mixing of the materials held behind each thread or die plate and the continual flow of additional materials.

In an embodiment of the present invention, the first carbohydrate and the second carbohydrate are different. In another embodiment of the present invention, the first and second carbohydrates may be the same carbohydrate in a different form if the different forms have different melting points. For example, the first carbohydrate may be in crystalline form while the second carbohydrate may be non-crystalline (e.g., amorphous). Altematively, one or both carbohydrates may be in a spray-dried form or granular form.

The present invention also includes a method for increasing the compactibility of a carbohydrate product. The method includes blending at least a first and a second carbohydrate, the second carbohydrate having a lower melting point temperature than the first carbohydrate, melting the second carbohydrate over the first carbohydrate, drying the product, and screening the product for the desired particle size. Desirable particle sizes range from about 50 microns to about 800 microns, more preferably about 75 microns to about 590 microns, and most preferably, about 100 microns to about 420 microns. In an embodiment of the present invention, the mean particle size is about 250 microns.

In an embodiment according to the present invention, the method includes blending at least a first and a second carbohydrate, each having a different melting point temperature, creating the highly compactible product by extruding the carbohydrate blend through an extruder, drying the extrudate, and screening the extrudate to a desired particle size. Desirable particle sizes range from about 50 microns to about 800 microns, more preferably about 75 microns to about 590 microns, and most preferably, about 100 microns to about 420 microns. In an embodiment of the present invention, the mean particle size is about 250 microns.

In another embodiment of the present invention, the first and second carbohydrate can be continuously fed individually to the extruder and blended during the first stage rather than being blended prior to addition to the extruder.

In another embodiment of the present invention, the first and second carbohydrate are blended prior to addition to hopper 1.

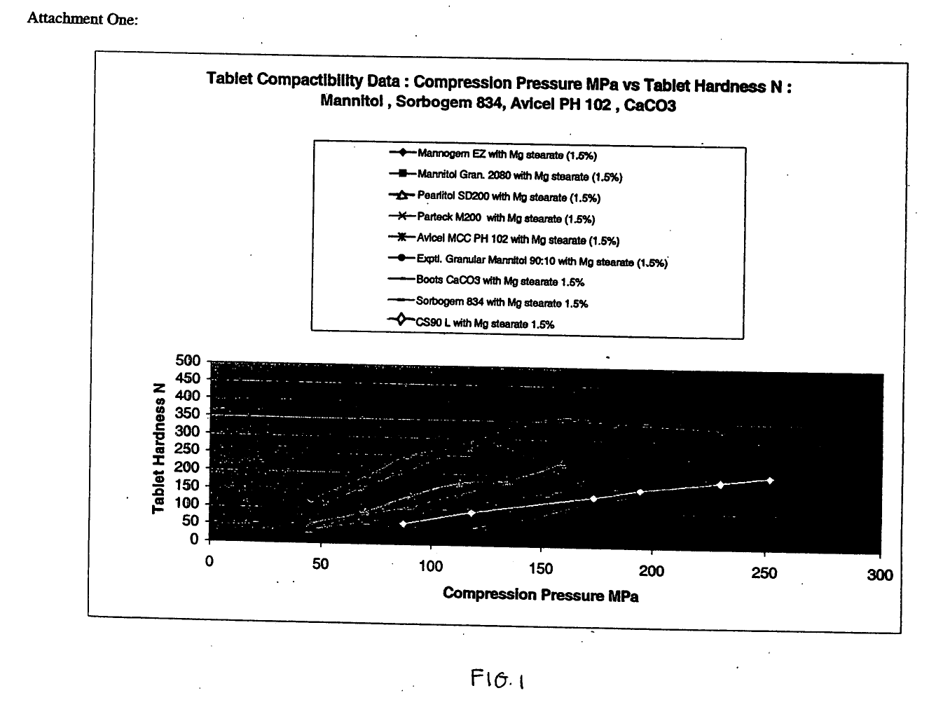

Without being bound to any particular theory, it is believed that the compactibility of a low compactibility carbohydrate can be enhanced by melting a second carbohydrate having a lower melting point over the first carbohydrate. FIG. 1 illustrates that at high compression forces, a composition according to the present invention (indicated by the black circle) maintains high tablet hardness. Changes in the surface characteristics of the first carbohydrate due in part to the underpressure flowability of the second carbohydrate contribute to the increased compactibility. The second carbohydrate (i.e., the lower melting point carbohydrate) is able to flow over and between the particles of the first carbohydrate (i.e., the higher melting point carbohydrate), creating a more uniformly coated first carbohydrate particle, and a more dense matrix of first carbohydrate particles. In one embodiment, the highly compactible product is useful for preparing, for example, a very robust pharmaceutical tablet because higher compactibility provides greater robustness.

Carbohydrates useful in the present invention include, but are not limited to sugars and polyols, which are sugar alcohols of the general formula CH₂OH—(CHOH)ₙ—CH₂OH, where n is 2 to 6, and preferably 3 to 6, and their dimeric anhydrides. Preferably, the polyols include, but are not limited to sorbitol, mannitol, erythritol, maltitol, lactitol, isomalt, and mixtures thereof, and sugars such as lactose, xylose, erythrose, fructose, dextrose, sucrose, maltose, and mixtures thereof. Preferred sugars include xylose, melted over maltose and xylose melted over sucrose.

In an embodiment according to the present invention, the raw carbohydrate materials are screened through a mesh screen. Mesh screen size can range from about 10 to about 80, preferably from about 20 to about 50, and more preferably about 20 mesh. In one embodiment, after screening, and prior to extrusion, the carbohydrates are mixed uniformly in a V-mixer, preferably 10 cubic feet (Patterson Kelley, East Stroudsburg, Pa.).

In one embodiment according to the present invention, after mixing the carbohydrates and screening them through an appropriately sized mesh screen, the mixture is compacted by extrusion through an extruder, such as the Reitz model RE-6 extruder (Hosokawa Bepex, Minneapolis, Minn.), at an rpm of from about 50 to about 120. The higher the content of the first carbohydrate, the higher the rpm necessary to extrude the product.

In an embodiment of the method of the present invention, the first and second carbohydrates have a minimum difference in melting temperature of from about 20 degrees Celsius to about 40 degrees Celsius, and preferably about 30 degrees Celsius. In one embodiment, the minimum melting temperature is preferably below 120 degrees Celsius, and more preferably below 110 degrees Celsius.

In one embodiment, the carbohydrate composition is extruded through two intermediate die plates (depicted in FIG. 4 as first die plate 2 and second die plate 3), for example, a one-half inch die plate internal to the unit, followed by a one-quarter inch die plate. In one embodiment, the composition is then extruded through an end die plate (depicted in FIG. 4 as end die plate 4), preferably a 12-gauge 0.047-inch 150 hole die plate (17.5% openings), as water is continually pumped into the water chamber of the extruder at a rate of about 100 cc/min. The water and the materials are heated to the melting point of the second carbohydrate such that the second carbohydrate melts and forms a solution with the water having a paste-like consistency. Other die plates are also useful in the present invention include any die plate that maintains the extrudate at a temperature of at least within about 10 percent above the melting point of the carbohydrate having the lower melting point.

The water content in the carbohydrate composition typically is equal to or less than about 3%, preferably less than 2%, and even more preferably less than 1%. The product is extruded through the die plate holes in the form of a noodle, wherein the second carbohydrate (i.e., the carbohydrate having a lower melting point) uniformly coats the first carbohydrate (i.e., the carbohydrate having a higher melting point).

The resulting “noodle” is dried in a fluid bed dryer (Fluid Air Model 1000, Aurora, Ill.) to a moisture content of less than 1%. The dried carbohydrate composition is screened and milled, for example, on a FITZMILL™ (Fitzpatrick D-6 Mill). In one embodiment, particle size may be from about 2000 to about 50 microns, corresponding to from about 10 to 80 mesh. The dried carbohydrate composition is directly compressible to form a tablet at this point.

In an embodiment of the present invention, the method proceeds as follows:

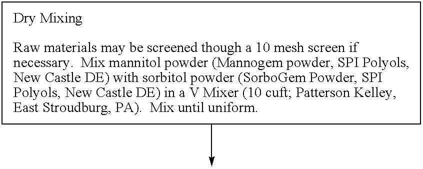

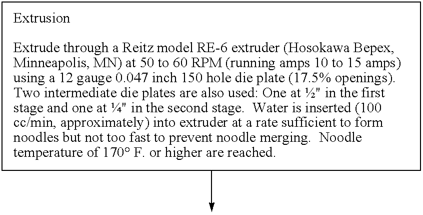

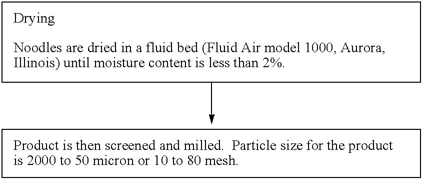

In one embodiment of the present invention, the method comprises blending mannitol (melting range between about 164 to 169 degrees C.) and sorbitol (melting range between about 95 and 97 degrees C.) in a 10 cubic foot blender (Patterson Kelley, East Stroudsburg, Pa.). The mannitol product can be from any source, and is preferably MANNOGEM ™ powder (SPI Polyols, Inc., New Castle, Del.). Other sources of mannitol powder include GETEC Mannitol powder (BRAZIL), and PEARLITOL™ (Roquette, FRANCE). Preferably, the form of mannitol is a platelike form of crystals, for example, the beta form of mannitol.

The sorbitol product can be from any source, and is preferably SORBOGEM™ powder (SPI Polyols, Inc., New Castle, Del.). Other sources of sorbitol include NeoSorb™ (Roquette, FRANCE), and Sorbitol Instant (Merck & Co., Whitehouse Station, N.J).

The ratio of mannitol:sorbitol can range from about 97:3 to about 70:30. Preferably, the mannitol to sorbitol ratio is about 80:20, and more preferably, about 90:10, and even more preferably about 97:3.

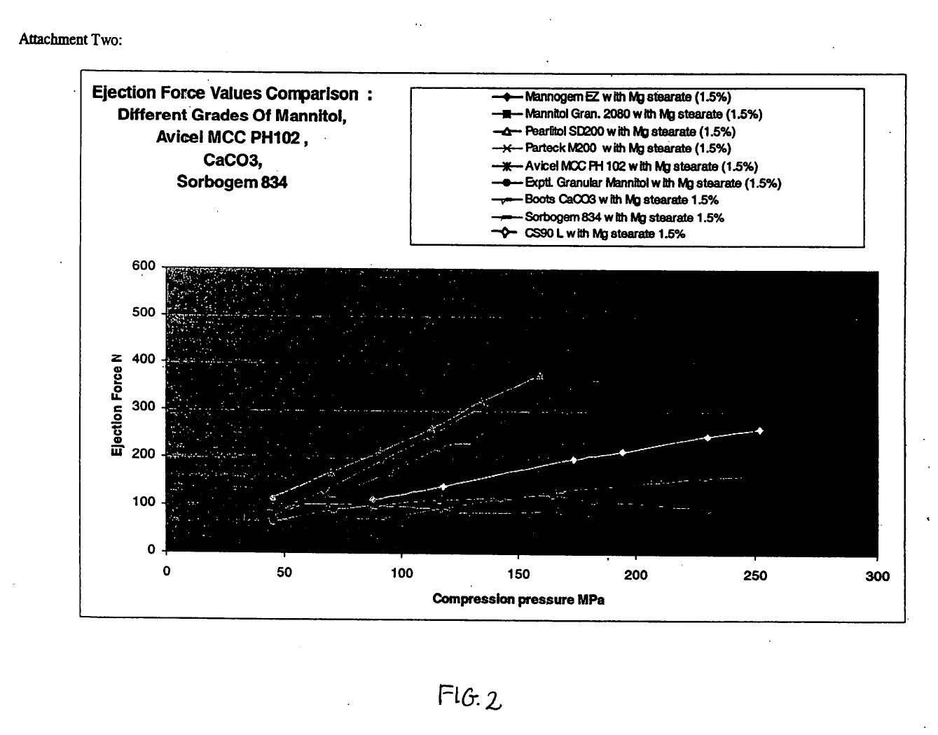

FIGS. 1 and 2 illustrate the superior results obtained with a mannitol/sorbitol composition discussed herein. A composition according to the present invention includes a 90:10 ratio of granular mannitol to crystalline sorbitol and about 1.5% magnesium stearate (indicated with a black circle on each graph). Each of the other compositions on the graph in FIGS. 1 and 2 is mannitol, a mannitol/sorbitol combination, or calcium carbonate in various forms are as follows:
(1) spray-dried mannitol with 1.5% magnesium stearate commercially available as MANNOGEM EZ (SPI Pharma, New Castle, Del.; solid diamond);
(2) spray-dried mannitol with 1.5% magnesium stearate commercially available as PEARLITOL SD200 Coquette, France; open triangle);
(3) granular mannitol with 1.5% magnesium stearate commercially available as Mannitol 2080 (SPI Pharma, New Castle, Del.; square)
(4) spray-dried mannitol with 1.5% magnesium stearate and 1.5% natural sorbitol impurities, commercially available as Parteck M200 (Merck & Co., Whitehouse Station, N.J.; “X”)
(5) Microcrystalline Cellulose with 1.5% magnesium stearate commercially available as AVICEL PH 102 (FMC Corp., Phila., Pa.; asterisk)
(6) boots calcium carbonate with 1.5% magnesium stearate (small rectangle)
(7) granular sorbitol commercially available as SORBOGEM 834 (SPI Pharma, New Castle, Del. large rectangle)
(8) Calcium Carbonate/Starch with 1.5% magnesium stearate commercially available as CS90 L (90:10 ratio calcium carbonate:starch, SPI Pharma, New Castle, Del.; open diamond).

The results on each of the graphs indicate that the composition of the present invention has high tablet hardness at relatively low compression forces, and low ejection forces compared with the other products tested. For example, at a compression force of 130 to 140 MPa, a composition of the present invention has sufficient tablet hardness of about 350 newtons (see FIG. 1). Only the Parteck M200 has a higher tablet hardness (about 375 newtons) at the same compression force. All other products tested had lower tablet hardness at the same compression force.

In addition, at the same compression force, a composition of the present invention has the lowest ejection force, at 100 newtons (see FIG. 2), compared with all other products tested. The ejection force for Parteck M200 is around 450 newtons at the same compression force. Therefore, the compositions of the present invention produce a tablet having a high tablet hardness and low ejection force at the same compression pressure, compared with all other products tested. This solves the problem of lamination during tableting.

It is notable that the compositions of the present invention follow similar compactibility and ejection force profiles to that of the SORBOGEM (black bar in FIGS. 1 and 2). This indicates that the surface of the highly compactible composition is sorbitol and the inner core is mannitol. Therefore, the compositions of the present invention enjoy the benefits of the compactibility and ejection force properties of sorbitol without the disadvantages of sorbitol, such as its hygroscopicity, decreased surface area, and increased viscosity, which are all poor characteristics for oral dosage forms. Mannitol/sorbitol compositions according to the present invention are preferable in oral dosage forms of pharmaceutical compositions because (1) these compositions have an increased surface area due to the mannitol, thereby making these compositions dissolve more quickly; (2) mannitol absorbs more calories when it S dissolves, thereby producing a cooling effect in the buccal cavity when the compositions dissolve; and (3) viscosity of mannitol is decreased in water, making the mannitol/sorbitol composition diffuse more quickly than a sorbitol composition.

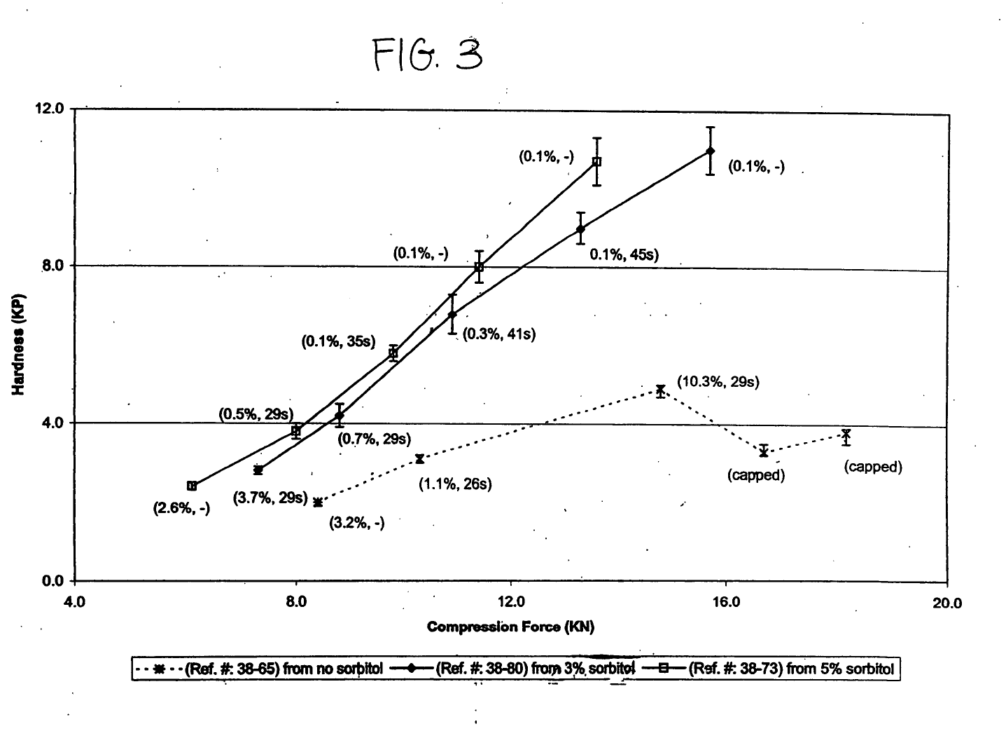

FIG. 3 is a graph representing another data set for compaction and ejection force profiles for compositions according to the present invention. The open boxes indicate a mannitol:sorbitol composition according to the present invention in a ratio of about 95:5. The black diamonds indicate a mannitol:sorbitol composition according to the present invention in a ratio of about 97:3. The asterisks indicate mannitol with less than 1% natural impurity sorbitol as a reference point. At each point on the graph, percent friability and disintegration time are noted in parentheses. The data indicate that tablet hardness for the 97:3 mannitol:sorbitol composition steadily increases even after the reference mannitol caps (i.e., the top of the tablet pops off). For example, at about 15 kilonewtons, tablet hardness for the 97:3 composition is about 11 KP, while tablet hardness for the reference mannitol is about 5 KP, and caps at a slightly higher compression force. In addition, the reference mannitol is about 100 times more friable than the 97:3 composition. Therefore, the data in this Figure indicate that a 97:3 mannitol:sorbitol composition has high compactibility (tablet hardness of about 10 KP), yet disintegrates in a short period of time (45 seconds) and has a very low friability (0.1%).

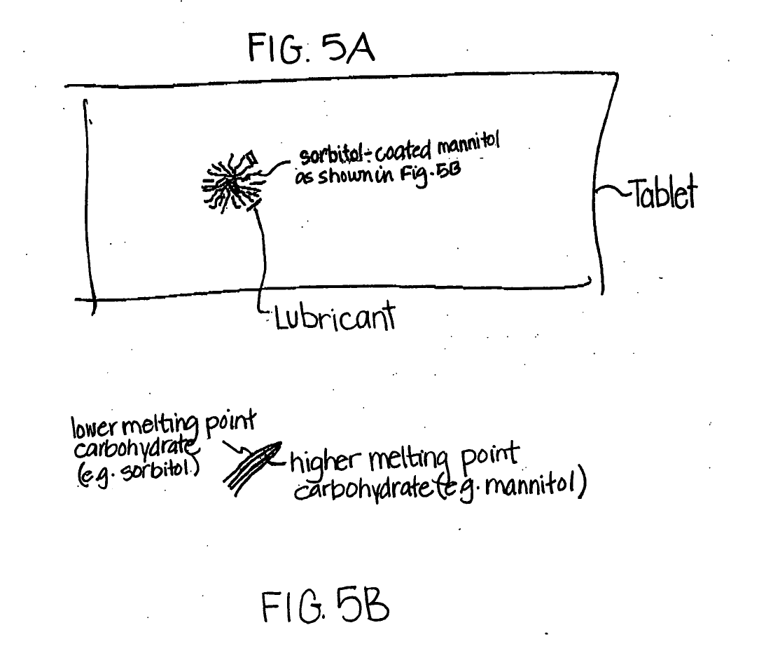

The present invention also includes a highly compactible carbohydrate composition produced by the methods discussed herein. The carbohydrate composition includes a first and a second carbohydrate, wherein the second carbohydrate is melted and coated uniformly over and between the particles of the first carbohydrate. The compositions of the present invention are novel in that they are made up of large particles having a large surface area. By way of example and not by limitation, a 100 micron particle in the composition of the present invention has the surface area characteristic of a 10 micron particle of a single polyol. The increased surface area is due to the fact that these particles are not solid, and internal spaces in the particles exist. A cross-section of a particle of the carbohydrate composition of the present invention, as depicted in FIGS. 5A and 5B, reveals that mannitol is coated with a layer of sorbitol, and the sorbitol-coated mannitol radiates out in the form of spikes from a more dense center. Lubricant is non-uniformly attached to the carbohydrate particle.

The present invention also includes a pharmaceutical composition. The pharmaceutical composition includes a highly compactible carbohydrate composition and one or more of at least one active ingredient, (e.g., calcium carbonate or acetaminophen), a lubricant, a color, and a flavor. The pharmaceutical composition can be in the form of a tablet, a capsule, a liquid, a film, or a gel. Preferably, the pharmaceutical composition is in the form of a tablet.

In one embodiment of the present invention, the pharmaceutical composition dissolves in the buccal cavity in about 60 seconds, preferably within about 45 seconds. In such embodiments, the highly compactible carbohydrate compositions have a mean particle size up to about 250 microns. The mean particle size of the compactible carbohydrate composition can be up to about 500 microns or more or as low as about 50 microns or more. Dissolution time is directly proportional to the mean particle size, such that as the mean particle size of a carbohydrate composition of the present invention increases, the dissolution time also increases.

In an embodiment of the present invention, the pharmaceutical composition includes the carbohydrate composition of the present invention in a range of from about 30 percent to about 99 percent by weight of the tablet.

Due to the high compactibility of a carbohydrate composition according to the present invention, the pharmaceutical composition typically requires less lubricant than a conventional pharmaceutical composition. The lubricant may be present in a pharmaceutical composition according to the present invention at about 0.1 percent to about 2 percent. Preferably, the lubricant is present at less than about 1 percent. Lubricants useful in the present invention include, but are not limited to sodium stearyl fumarate, glyceryl behenate, and magnesium stearate (“flow aids”). Lubricant attaches non-uniformly to the carbohydrate particles that make up the carbohydrate composition of the present invention.

In one embodiment, calcium carbonate is included as an active ingredient in a composition according to the present invention. Calcium carbonate is present in a range of from about 5 percent to about 40 percent, preferably from about 10 percent to about 30 percent, and more preferably about 20 percent.

In one embodiment, a sweetener may also be included in the composition of the present invention, and is preferably added to chewable tablets. Sweeteners may be present in a range of from about 0.01 percent to about 1 percent, preferably from about 0.05 percent to about 0.5 percent, and more preferably about 0.3 percent. Sweeteners useful in the present invention include, but are not limited to sucralose, aspartame, fructose, dextrose, dextrin, maltodextrin, corn syrup, high fructose corn syrup, saccharin, sucrose, acesulsame potassium, and glucose.

There is no limitation on color or flavor that is useful in the present invention, and these characteristics will likely be chosen based on the age of the patient consuming the pharmaceutical composition. Those of skill in the art will know which colors and flavors are useful in the present invention and the percent range of each present in the composition of the present invention. Color and flavor are inert ingredients and generally do not have any effect on the efficacy of the pharmaceutical composition.

In one embodiment of the composition according to the present invention, acetaminophen (APAP) is included as an active ingredient in a pharmaceutical composition according to the present invention. APAP is present in a range of from about 1 percent to about 30 percent, preferably from about 7 percent to about 25 percent, and more preferably about 14 percent.

In one embodiment of the present invention, a pharmaceutical composition according to the present invention includes:

| | |
|---|---|
| Carbohydrate composition | 62% |
| Calcium carbonate | 20% |
| 92% APAP | 14.5% |
| Flavor | 2% |
| Sweetener | 0.3% |
| Color | 0.3% |
| Lubricant | 1% |

Active ingredients useful in the composition of the present invention also include, but are not limited to pharmaceutical ingredients and nutraceutical ingredients. Examples of pharmaceutical ingredients that can be used include, but are not limited to gastrointestinal function conditioning agents, including, but not limited to bromopride, metoclopramide, cisapride, and domperidone; anti-inflammatory agents, including, but not limited to aceclofenac, diclofenac, flubiprofen, sulindac, and celecoxib; analgesics, including, but not limited to acetaminophen and aspirin; agents for erectile dysfunction therapy, including, but not limited to sildenafil and apomorphine; anti-migraines, including, but not limited to sumatriptan and ergotamin; antihistaminic agents, including, but not limited to loratadine, fexofenadine, pseudoephedrine and cetirizine; cardiovascular agents, including, but not limited to nitroglycerine and isosorbide dinitrate; diuretics, including, but not limited to furocemide and spironolactone; anti-hypertensive agents, including, but not limited to propranolol, amlodipine, felodipine, nifedipine, captoprile, ramiprile, atenolol, and diltiazem; anti-hypolipidemic agents, including, but not limited to simvistatin, atrovastatin, and pravastatin; anti-ulcer agents, including, but not limited to cimietidine, ranitidine, famotidine, omeprazole, and lansoprazol; anti-emetics, including, but not limited to meclizine hydrochoride, ondansetron, granisetron, ramosetron, and tropisetron; anti-asthmatic agents, including, but not limited to aminophylline, theophylline, terbuttaline, fenoterol, formoterol, and ketotifen; anti-depressants, including, but not limited to fluoxetine and sertraline; vitamins, including, but not limited to B1, B2, B6, B12 and C; anti-thrombotic agents, including, but not limited to sulfinpyrazone, dipyridamole, and ticlopidine; chemotherapeutic agents, including, but not limited to cefaclor, bacampicillin, sulfamethoxazole, and rifampicin; hormones, including, but not limited to dexamethasone and methyltestosterone; anti-helminthic agents, including, but not limited to piperazine, ivermectine, and mebendazole; and anti-diabetic agents, including, but not limited to acarbose, gliclazid, and glipizid.

Preferable pharmaceutical ingredients which may be used in the present invention include, but are non limited to acetaminophen, pseudoephedrine hydrochloride, dextromethorphan hydrobromide, dompereidone, famotidine, meclizine hydrochloride, scopolamine hydrobromide, ondansetron, cisapride, granisetron, sildenafil, loratadine, and amlodipine.

Examples of nutraceutical ingredients include, but are not limited to any ingredient that is thought to have a beneficial effect on human health. Such ingredients include coenzyme Q-10, chondroitoin, echinacea, ephedra, glucosamine, garlic, ginkgo biloba, ginseng, grape seed extract, guarana, hawthorn, herbs, kava, kola nut, lutein, St. John's wort, vinpocetine, and yohimbe.

It should be understood that the invention is not to be limited to the specific conditions or details described herein. Throughout the specification, any and all references to a publicly available document, including but not limited to a U.S. patent, are specifically incorporated by reference.

It will be apparent to those skilled in the art that various modifications and variations can be made in the methods and compositions of the present invention without departing from the spirit or scope of the invention. Thus, it is intended that the present invention cover the modifications and variations of the present invention provided they come within the scope of the appended claims and their equivalents.

What is claimed is:

1. A method for preparing a highly compactible carbohydrate product, said method comprising:
a) blending at least a first carbohydrate and a second carbohydrate, wherein the first carbohydrate has a melting point that is higher than the second carbohydrate;
b) melting the second carbohydrate over the first carbohydrate to obtain a highly compacted product;
c) drying the product; and
d) screening the dried product for desired particle size.

2. The method of claim 1, wherein said carbohydrates are selected from the group consisting of polyols and sugars.

3. The method of claim 2, wherein said polyols are selected from the group consisting of sorbitol, mannitol, erythritol, maltitol, lactitol, isomalt, and mixtures thereof.

4. The method of claim 2, wherein said sugars are selected from the group consisting of lactose, xylose, erythrose, fructose, dextrose, sucrose, maltose, and mixtures thereof.

5. The method of claim 1, wherein said first carbohydrate is mannitol and wherein said second carbohydrate is sorbitol.

6. The method of claim 5, wherein said mannitol is present in a range of from about 70 percent to about 97 percent, and wherein said sorbitol is present in a range of from about 3 percent to about 30 percent.

7. The method of claim 1, wherein in said step b, said second carbohydrate is melted over said first carbohydrate by extrusion.

8. The product resulting from the method of claim 1.

9. A carbohydrate composition, the composition comprising at least a first carbohydrate and a second carbohydrate, wherein said first carbohydrate has a melting point which is greater than said second carbohydrate, and said second carbohydrate is uniformly melted over said first carbohydrate.

10. The composition of claim 9, wherein said first carbohydrate is mannitol and said second carbohydrate is sorbitol.

11. The composition of claim 10, wherein said mannitol is present in a range of from about 70 percent to about 97 percent, and wherein said sorbitol is present in a range of from about 3 percent to about 30 percent.

12. The composition of claim 9 having a water content of about 1%.

13. The composition according to claim 9, further comprising a lubricant and an active ingredient.

14. The composition of claim 13, wherein said lubricant is magnesium stearate.

15. A method for preparing a highly compactible polyol product, said method comprising:
a) blending a first polyol and a second polyol, wherein said second polyol has a lower melting point than said first polyol;
b) extruding the polyol blend through an extruder;
c) drying the extrudate; and
d) screening the dried extrudate for desired particle size, wherein the temperature at said step b) reaches at least the melting point of said second polyol, but not said first polyol.

16. The method of claim 15, wherein said polyols are selected from the group consisting of sorbitol, mannitol, erythritol, maltitol, lactitol, isomalt, and mixtures thereof.

17. The method of claim 15, wherein said sugars are selected from the group consisting of lactose, xylose, erythrose, fructose, dextrose, sucrose, maltose, and mixtures thereof.

18. The method of claim 15, wherein said first polyol is mannitol and wherein said second polyol is sorbitol.

19. The method of claim 18, wherein said mannitol is present in a range of from about 70 percent to about 97 percent, and wherein said sorbitol is present in a range of from about 3 percent to about 30 percent.

20. The product resulting from the method of claim 15.

21. A method for preparing a highly compactible polyol product, said method comprising:
a) blending mannitol and sorbitol together in about a 97:3 ratio;
b) extruding the blend through an extruder, such that the temperature is high enough within said extruder to melt said sorbitol over said mannitol to create a highly compacted extrudate;
c) drying the extrudate; and
d) screening the dried extrudate for desired particle size.

22. The product resulting from the method of claim 21.

23. A method for increasing the compactibility of a polyol product, said method comprising:
a) blending a first polyol and a second polyol, wherein said second polyol has a lower melting point than said first polyol;
b) extruding the polyol blend through an extruder;
c) drying the extrudate; and
d) screening the dried extrudate for desired particle size, wherein the temperature at said step (b) reaches at least the melting point of said second polyol, but not said first polyol.
